(19) United States
(12) Patent Application Publication
(10) Pub. No.: US 2026/0151099 A1
(43) Pub. Date: Jun. 4, 2026

(54) DYNAMIC IMAGE PROCESSING APPARATUS, DYNAMIC IMAGE PROCESSING METHOD, AND STORAGE MEDIUM

(71) Applicant: KONICA MINOLTA, INC., Tokyo (JP)

(72) Inventors: Ryoichi WATANABE, Tokyo (JP); Kojiro ONO, Narita-shi (JP)

(73) Assignee: KONICA MINOLTA, INC., Tokyo (JP)

(21) Appl. No.: 19/349,504

(22) Filed: Oct. 3, 2025

(30) Foreign Application Priority Data
Oct. 16, 2024 (JP) 2024-180687

(51) Int. Cl. A61B 6/00; A61B 6/50; G06T 7/00

(52) U.S. Cl. CPC A61B 6/5217; A61B 6/507; A61B 6/5264; G06T 7/0012; G06T 2207/30104

(57) ABSTRACT

A dynamic image processing apparatus includes a hardware processor. The hardware processor generates waveform information including blood flow fluctuation and respiration-induced fluctuation in a predetermined region of each frame image constituting a dynamic image obtained by dynamic imaging by radiation. The hardware processor extracts the waveform information including dose fluctuation different from the blood flow fluctuation and the respiration-induced fluctuation from the generated waveform information.

### CROSS-REFERENCE TO RELATED APPLICATION

The entire disclosure of Japanese Patent Application No. 2024-180687, filed on Oct. 16, 2024, including description, claims, drawings and abstract is incorporated herein by reference.

### BACKGROUND OF THE INVENTION

### Technical Field

The present invention relates to a dynamic image processing apparatus, a dynamic image processing method, and a storage medium.

### Description of Related Art

Clinical research on dynamic analysis using dynamic X-ray dynamic images is underway, and the dynamic analysis is being utilized in examinations of various diseases. For example, a dynamic analysis method for analyzing blood flow such as pulmonary blood flow and cardiac blood flow using the X-ray dynamic image has been developed. Japanese U.S. Pat. No. 7,424,423 describes a dynamic image analysis apparatus that generates information on pulmonary valve regurgitation based on a dynamic image of a region of interest such as a pulmonary artery or a heart.

However, there are cases where noise is included in the dynamic image, because of body movement fluctuation due to unpredictable movement of a patient, coughing or the like, and respiratory movement fluctuation due to respiration, arrhythmia, or the like. However, with the conventional technology, although information on the pulmonary valve regurgitation and the like can be generated based on the dynamic image, the noise due to the body movement fluctuation and the like included in the dynamic image cannot be extracted. There is a problem that an appropriate analysis result cannot be obtained in a case in which dynamic analysis processing is performed by using the dynamic image including the noise due to the body movement fluctuation or the like.

Therefore, in order to solve the above-described problem, an object of the present invention is to provide a dynamic image processing apparatus, a dynamic image processing method, and a storage medium that can acquire dynamic images appropriate for dynamic analysis processing.

### SUMMARY OF THE INVENTION

According to one aspect of the present invention, a dynamic image processing apparatus according to an aspect of the present invention is the dynamic image processing apparatus including:
a hardware processor configured to,
generate waveform information including blood flow fluctuation and respiration-induced fluctuation in a predetermined region of each frame image constituting a dynamic image obtained by dynamic imaging by radiation, and
extract the waveform information including dose fluctuation different from the blood flow fluctuation and the respiration-induced fluctuation from the generated waveform information.

According to another aspect of the present invention, a dynamic image processing method according to an aspect of the present invention is the dynamic image processing method including:
generating waveform information including blood flow fluctuation and respiration-induced fluctuation in a predetermined region of each frame image constituting a dynamic image obtained by dynamic imaging by radiation, and
extracting the waveform information including dose fluctuation different from the blood flow fluctuation and the respiration-induced fluctuation from the generated waveform information.

According to another aspect of the present invention, a storage medium according to the present invention is a non-transitory computer-readable storage medium storing a program for causing a computer to perform,
generating waveform information including blood flow fluctuation and respiration-induced fluctuation in a predetermined region of each frame image constituting a dynamic image obtained by dynamic imaging by radiation, and
extracting the waveform information including dose fluctuation different from the blood flow fluctuation and the respiration-induced fluctuation from the generated waveform information.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present disclosure, and wherein:

FIG. 1 is a diagram illustrating an example of a schematic configuration of an image capturing system according to the present embodiment;

FIG. 2 is a diagram illustrating a frame image constituting a dynamic image in which a region of interest is set according to the present embodiment;

FIG. 3 is a dose waveform illustrating a change in a signal value of a pixel in the region of interest of the dynamic image according to the present embodiment;

FIG. 4 is a flowchart illustrating an example of an operation of a dynamic analysis apparatus in a case where noise due to body movement fluctuation is extracted from waveform information indicating dose fluctuation in the region of interest of the dynamic image according to the present embodiment;

FIG. 5 is a flowchart illustrating an example of the operation of a controller during a first processing according to the present embodiment;

FIG. 6 is a diagram illustrating a dose waveform in which a standard frame waveform is set according to the present embodiment;

FIG. 7 is a diagram illustrating the dose waveform in which a comparative frame waveform is set according to the present embodiment;

FIG. 8 is a flowchart illustrating an example of the operation of the controller during a second processing according to the present embodiment; and

FIG. 9 is a diagram illustrating the dose waveform of the region of interest of the dynamic image in which a first frame waveform, a second frame waveform, a third frame waveform, and a fourth frame waveform are set for each respiratory cycle according to the present embodiment.

### DETAILED DESCRIPTION

Hereinafter, one or more embodiments of the present disclosure will be described with reference to the drawings. However, the scope of the invention is not limited to the disclosed embodiments.

Below, with reference to the accompanying drawings, a dynamic image processing apparatus, a dynamic image processing method, and a storage medium according to preferred embodiments of the present disclosure are described in detail.

### Example of Configuration of Image Capturing System 100

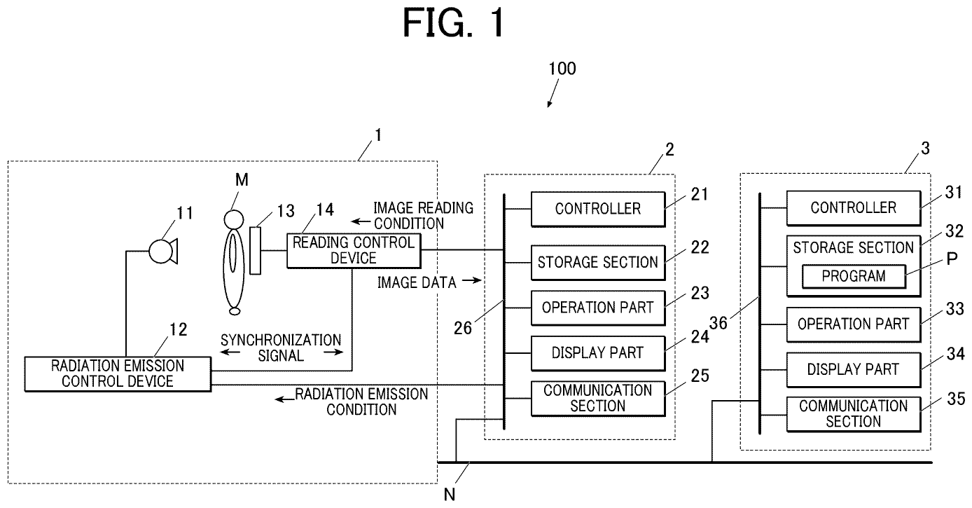

FIG. 1 is a diagram illustrating an example of a schematic configuration of an image capturing system 100 according to the present embodiment. The image capturing system 100 includes an imaging apparatus 1, a console 2, and a dynamic analysis apparatus 3 that is an example of a dynamic image processing apparatus. The imaging apparatus 1, the console 2, and the dynamic analysis apparatus 3 are communicably connected to each other via a network N such as a Local Area Network (LAN). A communication method of the network N may be wired communication or wireless communication.

The imaging apparatus 1 captures a dynamic image of a predetermined imaging area of a subject. The console 2 controls radiographic imaging by the imaging apparatus 1 and controls a reading operation of a radiographic image by the imaging apparatus 1. The dynamic analysis apparatus 3 performs predetermined dynamic analysis processing on the dynamic image transmitted from the console 2 or the like. According to the present embodiment, the dynamic analysis apparatus 3 extracts a frame waveform formed of a plurality of frame images including noise due to body movement fluctuation or the like of a subject M in a predetermined region of the dynamic image before performing the dynamic analysis processing. The apparatuses included in the image capturing system 100 comply with a Digital Image and Communications in Medicine (DICOM) standard, and communication between the apparatuses is performed in accordance with the DICOM standard.

### Example of Configuration of Imaging Apparatus 1

The imaging apparatus 1 can perform dynamic imaging of, for example, morphological changes in expansion and contraction of lungs due to respiratory motion and beating of a heart. The dynamic imaging refers to acquiring a series of images of the subject M by repeatedly irradiating the subject M with pulsed radiation such as X-rays at predetermined time intervals in response to one imaging operation. Repeatedly irradiating pulsed radiation at predetermined time intervals is referred to as pulsed irradiation. Alternatively, the dynamic imaging refers to acquiring a series of images of the subject M by continuously irradiating the subject M with a low dose rate without interruption in response to one imaging operation. Continuously applying radiation without interruption is referred to as continuous irradiation. The series of images obtained by dynamic imaging is called a dynamic image. Each of all of the images constituting the dynamic image is called a frame image. Here, the dynamic imaging includes moving image capturing, but does not include capturing a still image while displaying a moving image. Further, examples of a dynamic image include a moving image but do not include images obtained by capturing still images while displaying the moving image.

As shown in FIG. 1, the imaging apparatus 1 includes a radiation source 11, a radiation emission control device 12, a radiation detector 13, and a reading control device 14. The radiation emission control device 12 and the reading control device 14 are connected to each other via a communication cable or the like, and exchange synchronization signals with each other, thereby synchronizing a radiation emission operation and an image reading operation. The radiation detector 13 and the reading control device 14 may be integrally configured.

The radiation source 11 is arranged at a position facing the radiation detector 13 with the subject M interposed therebetween. The radiation source 11 irradiates the subject M with radiation such as X-rays under the control of the radiation emission control device 12. The radiation emission control device 12 is connected to the console 2. The radiation emission control device 12 controls the radiation source 11 on the basis of a radiation emission condition input from the console 2 to perform radiographic imaging. The radiation emission conditions input from the console 2 include, for example, a pulse rate, a pulse width, a pulse interval, the number of imaging frames per imaging, a value of an X-ray tube current, a value of an X-ray tube voltage, and a type of an additional filter. The pulse rate is the number of times that radiation is emitted per second, and matches a frame rate described below. The pulse width is a radiation irradiation time per radiation irradiation. The pulse interval is an amount of time from start of one radiation irradiation to start of the next radiation irradiation, and matches a frame interval described below.

The radiation detector 13 may include a semiconductor image sensor such as a flat panel detector (FPD). The FPD includes a substrate and the like formed of glass or the like. At predetermined positions on the substrate, a plurality of detection elements including pixels and the like are arranged in a matrix. The plurality of detection elements detect radiation emitted from the radiation source 11 and transmitted through at least the subject M in accordance with the intensity of the radiation, convert the detected radiation into an electrical signal, and accumulate the electrical signal. Each pixel includes a switching section such as a thin film transistor (TFT). Examples of a type of FPD include an indirect conversion type and a direct conversion type, and any type may be used. The indirect conversion type is a method in which the radiation is converted into the electrical signal by a photoelectric conversion element via a scintillator. The direct conversion type is a method of directly converting the radiation into the electrical signal.

The reading control device 14 is connected to the console 2. The reading control device 14 controls the switching section of each pixel of the radiation detector 13 based on an image reading condition input from the console 2. The reading control device 14 switches reading of the electrical signal accumulated in each pixel of the radiation detector 13, and acquires image data by reading the electrical signal accumulated in the radiation detector 13. The image data is each frame image of the dynamic image or a still image. If a structure exists between the radiation source 11 and the radiation detector 13, the amount of radiation reaching the radiation detector 13 decreases due to the structure. In this case, the signal value of each pixel of the image data changes according to the structure of the subject M. The signal value includes a pixel value, a density value, and the like. The reading control device 14 outputs the acquired dynamic image or still image to the console 2. The image reading condition includes, for example, a frame rate, a frame interval, a pixel size, and an image size. The frame rate is the number of frames acquired per second, and coincides with the pulse rate. The frame interval is the amount of time from the start of the operation of acquiring one frame image to the start of the operation of acquiring the next frame image, and matches with the pulse interval.

### Configuration Example of Console 2

The console 2 is, for example, a computer such as a personal computer, a workstation, or the like. As illustrated in FIG. 1, the console 2 includes a controller 21, a storage section 22, an operation part 23, a display part 24, and a communication section 25. The controller 21, the storage section 22, the operation part 23, the display part 24, and the communication section 25 are connected by wiring such as a bus 26.

The controller 21 includes a CPU (Central Processing Unit), a RAM (Random Access Memory), and the like. The CPU 21 reads a system program and various processing programs stored in the storage section 22 in response to an operation of the operation part 23, develops the programs in the RAM, and executes various processes in accordance with the developed programs. The controller 21 centrally controls operation of sections of the console 2 and the radiation emission operation and the reading operation of the imaging apparatus 1.

The storage section 22 is a nonvolatile semiconductor memory, a hard disk, or the like. The storage section 22 stores various programs to be executed by the controller 21, parameters required for execution of processing by the programs, and data such as processing results. The various programs are stored in the form of readable program codes. The controller 21 sequentially performs operations in accordance with the program codes.

The operation part 23 includes a keyboard, a mouse, and the like. The operation part 23 may be a touch screen combined with a display screen of the display part 24. The operation part 23 accepts various instructions by a user's input operation, and outputs an instruction signal corresponding to the accepted instruction to the controller 21.

The display part 24 is a monitor such as a liquid crystal display (LCD). The display part 24 displays an input instruction from the operation part 23, data, and the like in accordance with an instruction of a display signal input from the controller 21.

The communication section 25 includes a LAN adapter and a modem. The communication section 25 transmits and receives signals, data, and the like to and from the imaging apparatus 1, the dynamic analysis apparatus 3, and the like connected to the network N.

### Configuration Example of Dynamic Analysis Apparatus 3

The dynamic analysis apparatus 3 is used as a diagnosis support apparatus for supporting diagnosis by a doctor. The dynamic analysis apparatus 3 is constituted by, for example, a computer such as a personal computer or a workstation. As illustrated in FIG. 1, the dynamic analysis apparatus 3 includes a controller 31 (hardware processor), a storage section 32, an operation part 33, a display part 34, and a communication section 35. The controller 31, the storage section 32, the operation part 33, the display part 34, and the communication section 35 are connected by wiring of a bus 36.

The controller 31 includes a CPU, a RAM, and the like. The CPU reads various programs P such as a system program stored in the storage section 32 in response to an operation of the operation part 33, develops the programs in the RAM, and executes various processes in accordance with the developed programs. The controller 31 centrally controls the operation of each part of the dynamic analysis apparatus 3.

The storage section 32 includes a nonvolatile semiconductor memory, a hard disk, and the like. The storage section 32 stores various programs P to be executed by the controller 31, parameters required for execution of processing by the programs P, and data such as processing results. The various programs P are stored in the form of readable program codes. The controller 31 sequentially executes operations according to the program code.

The operation part 33 includes the keyboard, the mouse, and the like. The operation part 33 may be the touch screen combined with the display screen of the display part 24. The operation part 33 accepts various instructions by the user's input operation, and outputs the instruction signal corresponding to the accepted instruction to the controller 31.

The display part 34 includes the monitor such as an LCD. The display part 34 performs various displays in accordance with an instruction of a display signal input from the controller 31. The communication section 35 includes the LAN adapter and the modem. The communication section 35 transmits and receives signals, data, and the like to and from the console 2 and the like connected to the network N.

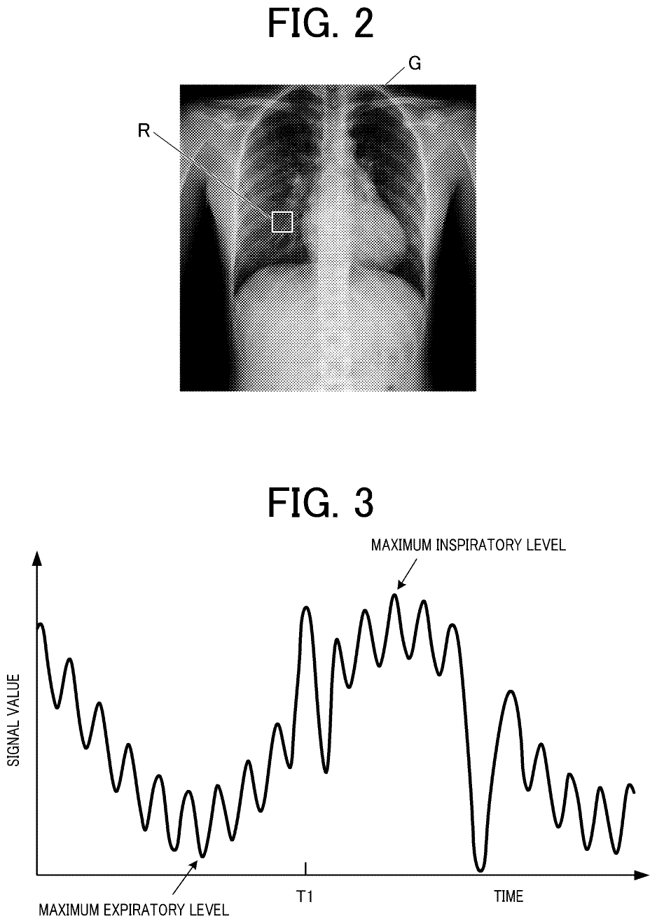

The controller 31 of the dynamic analysis apparatus 3 functions as a first generation section, an extraction section, a second generation section, and a setting section. The processor of the controller 31 realizes functions of the first generation section, the extraction section, the second generation section, the setting section, and the like by executing the program P stored in the storage section 32 or the like. The first generation section generates a dose waveform (waveform information) including a blood flow fluctuation and a respiration-induced fluctuation in a region of interest of each frame image constituting the dynamic image obtained by the dynamic imaging using the radiation. The region of interest is referred to as an ROI (Region of Interest). The dose waveform is generated based on the signal value of each pixel in the region of interest of each frame of the dynamic image. FIG. 2 is a diagram illustrating a frame image G constituting a dynamic image in which a region of interest R is set according to the present embodiment. The imaging area is a front of a chest. In the present embodiment, for example, the region of interest R is set in an area including the pulmonary artery in a lung field region of the frame image G. Thus, the user such as the doctor can diagnose pulmonary artery reflux.

The extraction section extracts, from the dose waveform generated by the first generation section, a frame waveform including a dose fluctuation different from the blood flow fluctuation and the respiration-induced fluctuation. Specifically, the extraction section compares the standard frame waveform in the waveform information with another frame waveform different from the standard frame waveform information. Hereinafter, the other frame waveforms are referred to as comparative frame waveforms. The standard frame waveform and the comparative frame waveform are a plurality of frame images and are composed of the same number of frames. When the standard frame waveform and the comparative frame waveform do not match, the extraction section extracts the comparative frame waveform as the frame image including noise such as body movement fluctuation. The random body movement fluctuation includes, for example, fluctuation due to unpredictable movement of the patient, coughing, and the like. The second generation section generates information indicating the dynamic image inappropriate for the dynamic analysis on the comparative frame waveform including the body movement fluctuation extracted by the extraction section. The setting section sets the standard frame waveform and the like based on the type of the dynamic analysis.

### Dose Waveform of Dynamic Image Region of Interest R

FIG. 3 is a dose waveform illustrating a change in the signal value of the pixel in the region of interest R of the dynamic image according to the present embodiment. In FIG. 3, a vertical axis represents the signal value of the pixel, and a horizontal axis represents time. The dose waveform is, for example, a graph obtained by averaging the signal values of the pixels in the region of interest R. The dose waveform includes at least the respiration-induced fluctuation associated with inhalation and exhalation of the subject M and a blood flow fluctuation associated with pulsation of the heart (heartbeat).

The respiration-induced fluctuation will be described. Air flows into a lung field during inhalation time from maximum exhalation to maximum inhalation. A maximum expiratory level is a timing at which the air in the lung field is fully exhaled. A maximum inspiratory level is the timing at which the air in the lung field is fully inhaled. In this case, the amount of transmitted X-rays is large in the lung field region, and the signal value of the pixel in the region of interest R increases from the maximum expiratory level toward the maximum inspiratory level. On the other hand, the air in the lung field flows out during an exhalation period from the maximum inhalation to the maximum exhalation. In this case, the amount of transmitted X-rays is small in the lung field region, and the signal value of the pixel in the region of interest R in the dynamic image decreases from the maximum inhalation toward the maximum exhalation. Therefore, as illustrated in FIG. 3, the respiration-induced fluctuation has a waveform that gradually changes between the maximum expiratory level and the maximum inspiratory level in accordance with inhalation and exhalation of the patient.

Subsequently, the blood flow fluctuation will be described. When the heart is in ventricular diastole, less blood flows into the lung field. In this case, the amount of transmitted X-rays increases in the pulmonary artery, and the signal value of each pixel in the region of interest R of the dynamic image also increases. On the other hand, when the heart is in ventricular systole, a large amount of blood flows into the lung field from the heart via the pulmonary artery. Therefore, the amount of transmitted X-rays decreases in the lung field region, and the signal value of each pixel in the region of interest R of the dynamic image also decreases. Therefore, as shown in FIG. 3, the blood flow fluctuation has a waveform in which the signal value repeatedly increases and decreases in accordance with the heartbeat of the heart, and is superimposed on the waveform of the respiration-induced fluctuation.

At the time of dynamic imaging, the random body movement fluctuation may occur due to unpredictable movement by the patient, coughing, poor breath holding, or the like. For example, in a case in which the random body movement fluctuation occurs in a vicinity of time T1 shown in FIG. 3, the waveform in the vicinity of the time T1 is largely displaced as compared with the waveform at the maximum inspiration level or the like at which the respiration-induced fluctuation is most stabilized. In a case where the waveform at the timing at which the random body movement fluctuation occurs is used for the dynamic analysis processing, an appropriate analysis result may not be obtained. Therefore, in the present embodiment, the frame waveform at a timing at which the random body movement fluctuation has occurred is extracted from a dose waveform, and information indicating that the extracted frame waveform is inappropriate for dynamic analysis processing is generated.

### Operation Example of Image Capturing System 100

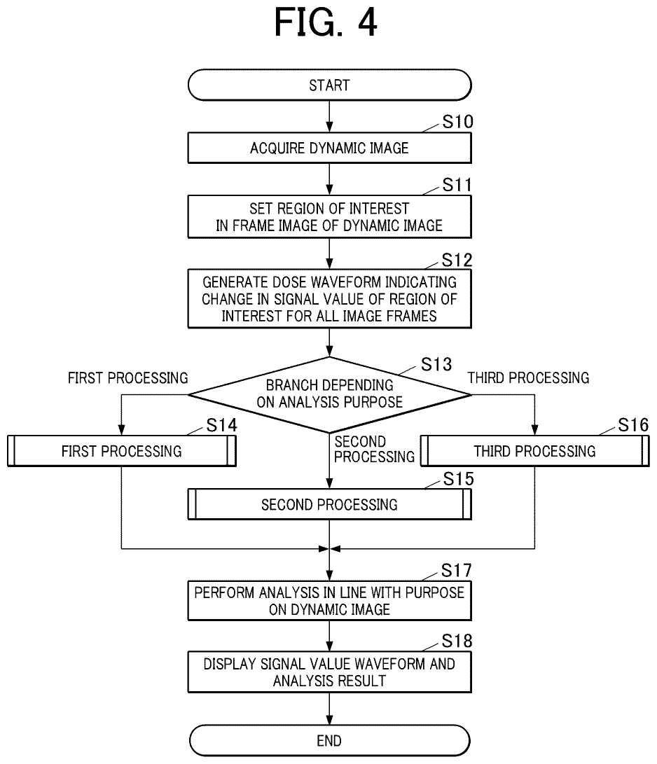

FIG. 4 is a flowchart illustrating an example of an operation of the dynamic analysis apparatus 3 according to the present embodiment in a case where the noise due to the body movement fluctuation is extracted from the waveform information indicating the dose fluctuation in the region of interest R of the dynamic image. The controller 31 implements processing including a generation step, an extraction step, and the like illustrated in FIG. 4 by executing the program P stored in the storage section 32.

First, operation of the imaging apparatus 1 and the console 2 during dynamic imaging will be described. The controller 21 of the console 2 sets the radiation emission condition in the radiation emission control device 12, and sets the image reading condition in the reading control device 14. Next, the controller 21 outputs an imaging start instruction of the dynamic image to the radiation emission control device 12 and the reading control device 14. The radiation source 11 of the imaging apparatus 1 irradiates the subject M with the radiation at the pulse interval set in the radiation emission control device 12. The reading control device 14 outputs the image data acquired by the radiation detector 13 to the console 2.

The controller 21 of the console 2 stores each frame image of the dynamic image included in the image data transmitted from the imaging apparatus 1 in the storage section 22 in association with the frame number indicating the imaging order. Next, the controller 21 allows the display part 24 to display the acquired dynamic image. The user such as a radiologist checks whether the image is suitable for diagnosis. Based on a confirmation instruction from the user, the controller 21 adds patient information, examination information, and the like to the dynamic image acquired by the dynamic imaging, and transmits the dynamic image to the dynamic analysis apparatus 3.

As illustrated in FIG. 4, the controller 31 of the dynamic analysis apparatus 3 acquires the dynamic image of a predetermined imaging area from the console 2 via the communication section 35 (step S10). For example, the imaging area is the front of the chest illustrated in FIG. 2. The dynamic image is composed of a plurality of frame images. The controller 31 stores the acquired dynamic image in the storage section 32.

The controller 31 sets the region of interest R in the frame image of the acquired dynamic image (step S11). For example, as illustrated in FIG. 2, the controller 31 sets the region of interest R in the vicinity of the pulmonary artery in the lung field. The vicinity of the pulmonary artery is a portion with a large blood flow and appears whitish in the dynamic image. Therefore, the controller 31 can automatically set, as the region of interest R, the region in which many pixels having high luminance values are gathered in the lung field region in the dynamic image. Note that the setting of the region of interest R is not limited to the automatic setting by the controller 31. The user such as the radiologist may manually set the region of interest R. In this case, for example, the display part 34 may be caused to display the image of the lung field region in which a contrast between a portion that looks white due to the blood flow and the other portion is illustrated in an easily understandable manner. The user can set the region of interest R by selecting the vicinity of the pulmonary artery in the image displayed on the display part 34 with the operation part 33.

The controller 31 generates the radiation dose waveform indicating the change in the signal value of each pixel in the region of interest R for all the set frame images (step S12). Specifically, as illustrated in FIG. 3, the controller 31 generates the dose waveform including the respiration-induced fluctuation due to inspiration and expiration and the blood flow fluctuation due to the pulsation of the heart (heartbeat) in the region of interest R of each frame image of the dynamic image.

One of the first process, the second process, and the third process is performed on the generated radiation dose waveform indicating the change in the signal value according to the analysis purpose before the dynamic analysis processing (step S13). For example, the user may select the process suitable for the analysis purpose of the dynamic image from the items of the first to third processes displayed on the screen of the display part 34 by operating the operation part 33. In addition, the controller 31 may automatically acquire processing suitable for the analysis purpose of the acquired dynamic image on the basis of information such as the imaging area and the region of interest. Here, the first processing is processing for extracting the frame waveform including a fluctuation corresponding to the random body motion from the dose waveform. The second processing is processing for extracting a period having a small influence on periodic noise from the dose waveform. The third process is a process for extracting the frame image including the body motion based on the standard frame image in a case where the region of interest R is the entire image.

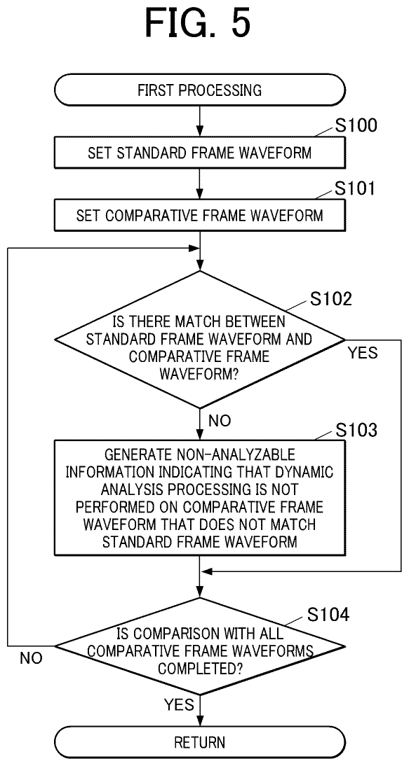

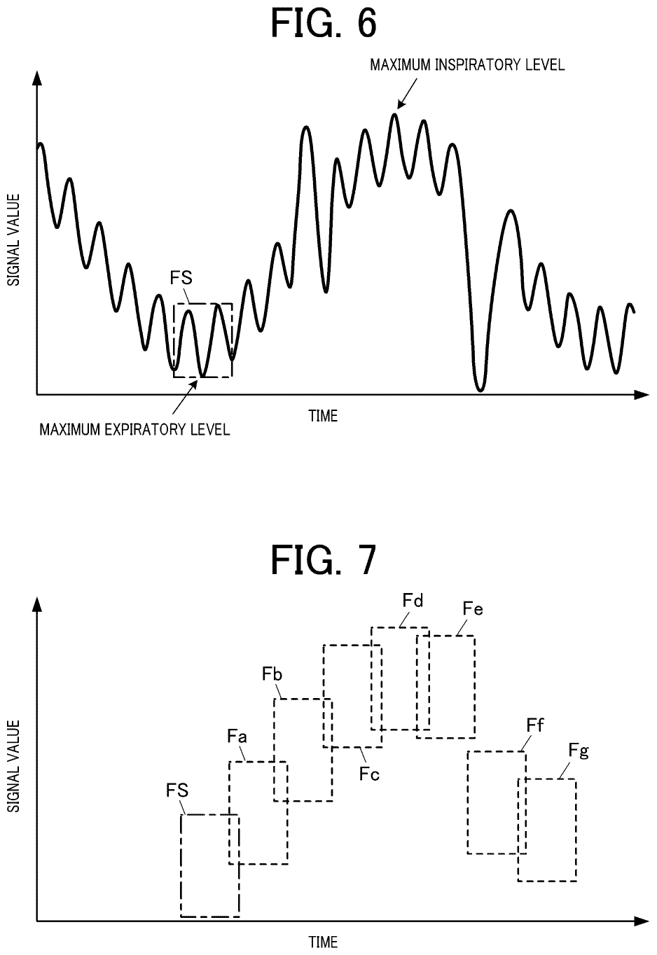

When the process branches to the first process in step S13, the controller 31 performs the first process on the acquired dynamic image (step S14). In this case, the controller 31 proceeds to a subroutine of FIG. 5. FIG. 5 is a flowchart illustrating an example of the operation of the controller 31 during the first process according to the present embodiment. As illustrated in FIG. 5, the controller 31 sets the standard frame waveform in the dose waveform of the region of interest in each frame image of the dynamic image (step S100). FIG. 6 is a view showing the dose waveform in which the standard frame waveform FS according to the present embodiment is set. The standard frame waveform FS may be constituted by, for example, a plurality of frame images of the maximum expiratory level and the vicinity thereof, or may be constituted by a plurality of frame images of the maximum inspiratory level and the vicinity thereof. This is because the frame images at the maximum expiratory level and the maximum inspiratory level are least affected by the respiration-induced fluctuation, and when used in the dynamic analysis processing, an appropriate result of the dynamic analysis processing can be obtained. In the present embodiment, the standard frame waveform FS is set using a plurality of frame images at the maximum expiratory level and in the vicinity thereof. The standard frame waveform FS is set so as to include, for example, peaks corresponding to two heartbeats due to the blood flow fluctuation. In FIG. 6, a range including the standard frame waveform FS is indicated by a rectangular frame of a one dot chain line.

The controller 31 may set the standard frame waveform FS in accordance with the type of dynamic analysis to be performed. This is because the content of the dynamic analysis is different depending on the type of the dynamic analysis, and the extent to which the noise such as the body movement fluctuation affects each dynamic analysis is also different. The type of dynamic analysis can be determined on the basis of an imaging condition such as an imaging area, for example. The imaging condition such as the imaging area is set based on the order information transmitted from the RIS or the like.

The controller 31 sets the comparative frame waveform to be compared with the standard frame waveform FS in the radiation dose waveform (step S101). FIG. 7 is a view showing the dose waveform in which a comparative frame waveform Fa and the like according to the present embodiment are set. The controller 31 may sequentially set the comparative frame waveform Fa and the like by, for example, moving the rectangular frame of the standard frame waveform FS along a time direction of the dose waveform. Specifically, when the standard frame waveform FS is in the 50 th to 60 th frames, the comparative frame waveforms Fa and the like can be sequentially set by moving the standard frame waveform FS every five frames in one example. In this case, the comparative frame waveform Fa is the 55 th to 65 th frames. Note that the number of frames to be moved is not limited to five frames and may be, for example, one frame. In FIG. 7, a range including the comparative frame waveforms Fa and Fb is indicated by a rectangular frame of a broken line. Note that the number of comparative frame waveforms set is not limited to the number illustrated in FIG. 7. Alternatively, the user may manually set the standard frame waveform FS, the comparative frame waveform Fa, and the like while checking the screen of the display part 34.

The controller 31 sequentially determines whether the standard frame waveform FS and the comparative frame waveform Fa or the like match each other (step S102). Specifically, the controller 31 compares the standard frame waveform FS with the comparative frame waveform Fa or the like, and determines the similarity between these frame waveforms. The controller 31 may determine the similarity to the comparative frame waveform Fa or the like using, for example, the number of peaks, the signal value, or the like of the standard frame waveform FS. For example, when determining the similarity using signal values, the controller 31 can determine that the similarity between the standard frame waveform FS and the comparative frame waveform is high when the amplitude of the comparative frame waveform is in a range of 90% to 110% with respect to the amplitude of the standard frame waveform FS being 100%. Further, the determination condition of the similarity may be a condition other than the number of peaks of the standard frame waveform FS and the width of the signal value, and may be, for example, a cross-correlation function using the entire signal value waveform.

Specifically, when the comparison target with the standard frame waveform FS is the comparative frame waveform Fa, the similarity is determined as follows. As illustrated in FIG. 7, the number of peaks of the standard frame waveform FS and the number of peaks of the comparative frame waveform Fa are two and coincide with each other within the rectangular frame. Therefore, the controller 31 can determine that the similarity between the standard frame waveform FS and the comparative frame waveform Fa is high. In this case, the controller 31 determines that the standard frame waveform FS and the comparative frame waveform Fa match each other, and proceeds to step S104.

When the comparison target with the standard frame waveform FS is the comparative frame waveform Fb, the similarity is determined as follows. As illustrated in FIG. 7, the number of peaks of the standard frame waveform FS is two within the rectangular frame, and the number of peaks of the comparative frame waveform Fb is unknown within the rectangular frame. Therefore, the controller 31 can determine that the similarity between the standard frame waveform FS and the comparative frame waveform Fb is low. The controller 31 extracts the comparative frame waveform Fb as the frame waveform including the body motion or the like of the patient. In this case, the controller 31 determines that the standard frame waveform FS and the comparative frame waveform Fa do not match, and proceeds to step S103.

The controller 31 generates non-analyzable information indicating that the comparative frame waveform Fb or the like that does not match the standard frame waveform FS is inappropriate for the dynamic analysis processing (step S103). Specifically, processing for a case where the dynamic image includes 100 frame images and 80 th to 90 th frame images include the noise due to the body movement fluctuation is executed as follows. In this case, the controller 31 generates the non-analyzable information for the 80 th to 90 th frame images and adds the generated non-analyzable information to each of the 80 th to 90 th frame images. After generating the non-analyzable information, the controller 31 proceeds to step S104.

The controller 31 determines whether the comparison of all the set comparative frame waveforms Fa and the like has been completed (step S104). When determining that the comparison of all the set comparative frame waveforms Fa and the like has not been completed, the controller 31 returns to step S102. The controller 31 moves the comparison target with the standard frame waveform FS to the adjacent comparative frame waveform or the like, and repeatedly executes the above-described comparison processing of the standard frame waveform FS. On the other hand, when determining that the comparison with all the set comparative frame waveforms Fa and the like has been completed, the controller 31 proceeds to step S17 illustrated in FIG. 4.

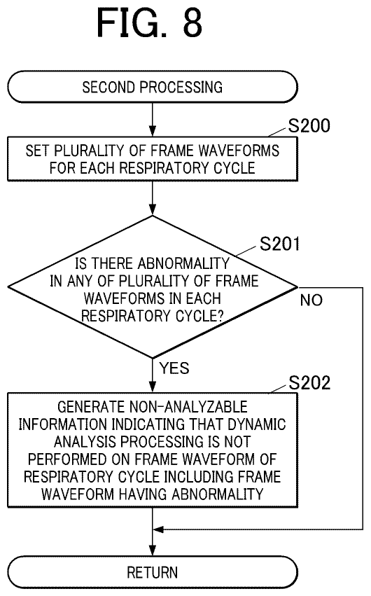

When the process branches to the second process in step S13 of FIG. 4, the controller 31 performs the second process on the acquired dynamic image (step S15). In this case, the controller 31 proceeds to the subroutine of FIG. 8. FIG. 8 is a flowchart illustrating an example of the operation of the controller 31 during the second process according to the present embodiment.

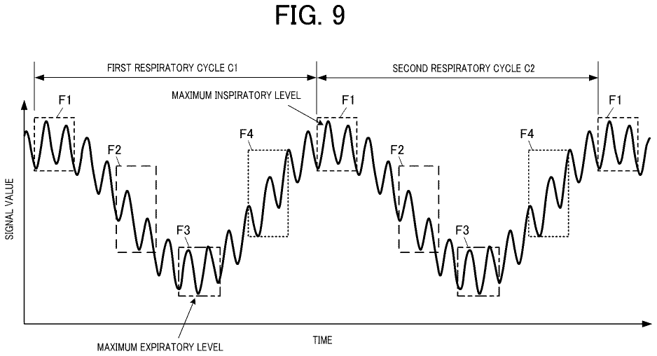

The controller 31 sets a plurality of frame waveforms in each respiratory cycle in the radiation dose waveform (step S200). FIG. 9 is a diagram showing a radiation dose waveform in which a first frame waveform F1, a second frame waveform F2, a third frame waveform F3, and a fourth frame waveform F4 are set in each respiratory cycle according to the present embodiment. In the first respiratory cycle C1, the controller 31 sets the first frame waveform F1 in the maximum inspiration period, sets the second frame waveform F2 in the expiration period, sets the third frame waveform F3 in the maximum expiration period, and sets the fourth frame waveform F4 in the inspiration period. The controller 31 sets the first frame waveform F1, the second frame waveform F2, the third frame waveform F3, and the fourth frame waveform F4 in the same manner as the first respiratory cycle C1 in the second respiratory cycle C2 and the subsequent respiratory cycles.

Here, the first frame waveform F1 is constituted by a plurality of frame images in the maximum inspiration period. The second frame waveform F2 is constituted by a plurality of frame images at a substantially intermediate position between the maximum inspiratory level and the maximum expiratory level. The third frame waveform F3 is constituted by a plurality of frame images in the maximum exhalation period. The fourth frame waveform F4 is constituted by a plurality of frame images at a substantially intermediate position between the maximum expiratory level and the maximum inspiratory level. Note that the number of frame waveforms to be set is not limited to that in FIG. 9. Furthermore, the setting position of the frame waveform is also not limited to FIG. 9 and can be set at an arbitrary position (period) of the dose waveform.

The controller 31 compares frame waveforms in the same period set for each respiratory cycle. Based on the comparison result, the controller 31 determines, for each respiratory cycle, whether any of the plurality of frame waveforms in each respiratory cycle has an abnormality (step S201). To be specific, as shown in FIG. 9, the controller 31 compares the corresponding first frame waveforms F1, the corresponding second frame waveforms F2, the corresponding third frame waveforms F3, and the corresponding fourth frame waveforms F4, which are set for every n respiratory cycles. The value n is a positive integer. The controller 31 may determine the similarity between the frame waveforms on the basis of the number of peaks in each frame waveform, the width of the signal value of the waveform, or the like.

When the frame waveform having a low degree of similarity to the corresponding frame waveform of another respiratory cycle is not included in the first frame waveform F1 or the like of the first respiratory cycle C1, the controller 31 determines that the periodic noise is not included in the first respiratory cycle C1. The controller 31 performs the same process as the first respiratory cycle C1 for other respiratory cycles other than the first respiratory cycle C1. When the periodic noise is not included in all the respiratory cycles, the controller 31 determines that there is no abnormality in all the respiratory cycles. In this case, the controller 31 proceeds to step S17 in FIG. 4.

On the other hand, when the frame waveform having the low degree of similarity to the corresponding frame waveform of another respiratory cycle is included in the first frame waveform F1 or the like of the first respiratory cycle C1, the controller 31 determines that the periodic noise is included in the first respiratory cycle C1. The controller 31 performs the same process as the first respiratory cycle C1 for other respiratory cycles other than the first respiratory cycle C1. When the periodic noise is included in at least one or more respiratory cycles, the controller 31 determines that there is the abnormality in any of the respiratory cycles. In this case, the controller 31 extracts the respiratory cycle including the periodic noise, and proceeds to step S202.

The controller 31 generates non-analyzable information indicating that the extracted frame waveform of the respiratory cycle having the abnormality is inappropriate for the dynamic analysis processing (step S202). For example, the controller 31 may extract the respiratory cycle with little periodic noise by adding the non-analyzable information to all frame waveforms of the specified respiratory cycle. After generating the non-analyzable information, the controller 31 proceeds to step S17 in FIG. 4.

In the case of branching to the step S13 of FIG. 4, the controller 31 executes the third processing on the acquired dynamic image (step S16). Specifically, the controller 31 determines, as the standard frame image, the frame image of a normal portion assumed not to include the body motion in the signal value waveform in a case in which the region of interest R of each frame is set as the entire image. The controller 31 calculates the similarity to the standard frame image in each frame of the specific number of frames to be used for the dynamic analysis. The similarity may be determined using the signal value or the like, as described above. Based on the comparison result of the similarity, the controller 31 determines that the frame image whose similarity is low and exceeds the threshold value is an abnormal frame image including the noise due to body motion, and extracts the frame image whose similarity is low. The controller 31 may reject the extracted frame image, or may add non-analyzable information to the extracted frame image as described above.

The controller 31 executes the dynamic analysis processing according to the purpose using the generated dose waveform (step S17). In a case where the first process and the second process are executed, the controller 31 executes the dynamic analysis processing on the frame image in which the non-analyzable information is not added to the dynamic image. That is, the controller 31 executes the dynamic analysis processing on the frame image not including the noise due to the patient's body motion or the like.

The controller 31 causes the display part 34 to display the generated dose waveform and the dynamic analysis result on the screen (step S18). For example, in a case where the first process and the second process are executed, the controller 31 displays the result of the dynamic analysis processing executed on the frame image to which the non-analyzable information is not added on the screen of the display part 34. At this time, the controller 31 may display, in a pop-up window on the screen of the display part 34, a message indicating that the frame image to which the non-analyzable information has been added is not being used for the dynamic analysis processing.

According to the present embodiment, by performing the first process, the dynamic analysis apparatus 3 compares the standard frame waveform and the comparative frame waveform in the dose waveform, and extracts the comparative frame waveform that does not match the standard frame waveform. As a result, it is possible to extract the frame waveform including the noise due to the random body movement fluctuation of the patient from the respiratory cycle of the dose waveform. In addition, the dynamic analysis apparatus 3 uses, as the standard frame waveform, the frame image of the maximum inspiration period or the maximum expiration period, which is the timing at which the respiration-induced fluctuation settles. Therefore, since only the frame image of the portion with little respiration-induced fluctuation can be extracted from the dose waveform, a high accuracy dynamic image not including the random body movement fluctuation can be obtained. Accordingly, since a predetermined dynamic analysis can be performed using the appropriate dynamic image, it is possible to obtain the accurate analysis result.

According to the present embodiment, when the plurality of frame waveforms set for each respiratory cycle include the frame waveform having low similarity, the dynamic analysis apparatus 3 can determine, by performing the second processing, that the respiratory cycle includes the periodic noise such as involuntary movement of a neurological symptom and irregular heartbeat. In addition, since the dynamic analysis apparatus 3 adds the non-analyzable information to the frame waveform of the respiratory cycle including the periodic noise, it is possible to obtain the dynamic image with high accuracy. Accordingly, since a predetermined dynamic analysis can be performed using the appropriate dynamic image, it is possible to obtain the accurate analysis result.

Although the preferred embodiments of the present disclosure have been described in detail with reference to the accompanying drawings, the technical scope of the present disclosure is not limited to such examples. Furthermore, those to which various modification examples and improvements have been applied naturally belong to the technical scope of the present disclosure within the category of the technical idea described in the scope of the claims of those skilled in the art.

For example, although the dynamic analysis apparatus 3 performs the extraction processing of the noise such as the random body movement fluctuation of the patient included in the dynamic image in the above embodiment, it is not limited thereto. For example, the console 2 may function as the dynamic image processing apparatus and perform extraction processing of the noise included in the dynamic image. An information processing apparatus such as a client terminal may function as the dynamic image processing apparatus and perform processing for extracting the noise included in the dynamic image.

Furthermore, although the process of extracting the noise included in the dynamic image is performed by using the operation part 33 and the display part 34 of the dynamic analysis apparatus 3 of the above-described embodiment, it is not limited thereto. For example, the operation part and the display part of the information processing apparatus in another client terminal or the like connected to the network N may be used. In this case, the dynamic analysis apparatus 3 may not include the operation part 33 and the display part 34.

Although embodiments of the present disclosure have been described and illustrated in detail, the disclosed embodiments are made for purposes of illustration and example only and not limitation. The scope of the present disclosure should be interpreted by terms of the appended claims.

What is claimed is:

1. A dynamic image processing apparatus comprising:
a hardware processor configured to,
generate waveform information including blood flow fluctuation and respiration-induced fluctuation in a predetermined region of each frame image constituting a dynamic image obtained by dynamic imaging by radiation, and
extract the waveform information including dose fluctuation different from the blood flow fluctuation and the respiration-induced fluctuation from the generated waveform information.

2. The dynamic image processing apparatus according to claim 1, wherein the hardware processor generates information indicating the dynamic image inappropriate for dynamic analysis with respect to the extracted waveform information.

3. The dynamic image processing apparatus according to claim 1, wherein the hardware processor extracts, based on a comparison between first waveform information serving as a standard in the waveform information and second waveform information different from the first waveform information, the second waveform information in a case where the first waveform information and the second waveform information do not match.

4. The dynamic image processing apparatus according to claim 3, wherein the first waveform information includes a plurality of frame images in a maximum respiratory cycle or a plurality of frame images in a maximum inspiration period due to the respiration-induced fluctuation.

5. The dynamic image processing apparatus according to claim 3, wherein the hardware processor sets the first waveform information based on a type of dynamic analysis.

6. The dynamic image processing apparatus according to claim 3, wherein the hardware processor extracts the second waveform information in a case where the second waveform information includes a body movement fluctuation of a patient.

7. The dynamic image processing apparatus according to claim 1, wherein the hardware processor generates the waveform information based on a signal value of each pixel in the predetermined region of each frame image of the dynamic image.

8. A dynamic image processing method comprising:
generating waveform information including blood flow fluctuation and respiration-induced fluctuation in a predetermined region of each frame image constituting a dynamic image obtained by dynamic imaging by radiation, and
extracting the waveform information including dose fluctuation different from the blood flow fluctuation and the respiration-induced fluctuation from the generated waveform information.

9. A non-transitory computer-readable storage medium storing a program for causing a computer to perform,
generating waveform information including blood flow fluctuation and respiration-induced fluctuation in a predetermined region of each frame image constituting a dynamic image obtained by dynamic imaging by radiation, and
extracting the waveform information including dose fluctuation different from the blood flow fluctuation and the respiration-induced fluctuation from the generated waveform information.
